(19) United States
(12) Patent Application Publication
(10) Pub. No.: US 2025/0057877 A1
(43) Pub. Date: Feb. 20, 2025

(54) MULTIPLE USE WOUND AND HEALING CREAM

(71) Applicants: Terris Viola Coleman, Sacramento, CA (US); Terris Christina Coleman, Sacramento, CA (US)

(72) Inventors: Terris Viola Coleman, Sacramento, CA (US); Terris Christina Coleman, Sacramento, CA (US)

(21) Appl. No.: 18/233,535

(22) Filed: Aug. 14, 2023

(51) Int. Cl. A61K 33/30; A61K 9/06; A61K 38/12

(52) U.S. Cl. CPC A61K 33/30; A61K 9/06; A61K 38/12

(57) ABSTRACT

The present disclosure relates to a formulation including a balm, cream, or salve including petroleum, lanolin, paraffin wax, or 8-hydroxyquinoline sulfate 0.3%. The formulation may include an oxide constituent and an antibiotic. The present disclosure also relates to a method of administering a formulation including a balm, cream, or salve and an oxide constituent, and an antibiotic.

### TECHNICAL FIELD

The embodiments generally relate to formulations of healing creams, salves, or balms and methods of healing wounds via topical cream, balm, or the like.

### BACKGROUND

Formulations and methods of administering formulations designed for treatment and healing of external wounds such as, but not limited to burns, abrasion, laceration, puncture, or avulsion may include various constituents for the reduced chance of bacterial infection, increased healing rate, and scar reduction.

### SUMMARY OF THE INVENTION

This summary is provided to introduce a variety of concepts in a simplified form that is further disclosed in the detailed description of the embodiments. This summary is not intended to identify key or essential inventive concepts of the claimed subject matter, nor is it intended for determining the scope of the claimed subject matter.

The present disclosure relates to a formulation including a balm, cream, or salve and an oxide constituent, and an antibiotic.

The present disclosure relates to a method of administering a formulation including a balm, cream, or salve and an oxide constituent, and an antibiotic.

Both the foregoing general description and the following detailed description present embodiments intended to provide an overview or framework for understanding the nature and character of the embodiments disclosed herein. Any accompanying drawings are included to provide further understanding and are incorporated into and constitute a part of this specification. The drawings illustrate various embodiments of the disclosure, and together with the description explain the principles and operations thereof.

### BRIEF DESCRIPTION OF THE DRAWINGS

A complete understanding of the present embodiments and the advantages and features thereof will be more readily understood by reference to the following detailed description when considered in conjunction with the accompanying drawings wherein:

FIG. 1 depicts a flowchart of a method of administering a formulation as described herein.

### DETAILED DESCRIPTION

The specific details of single embodiments or variations described herein are to the described formulation and methods of use. Any specific details of the embodiments are used for demonstration purposes only and no unnecessary limitations or inferences are to be understood from there.

According to various embodiments illustrating the practice of the present method and formulation, a person having ordinary skill in the art will understand that the method and formulation may be practiced or applied according to other variant embodiments. Many other possible modifications and variations of the details of the present formulation and method based on different outlooks and applications may be made without departing from the spirit of this disclosure.

According to some embodiments, a multi-use healing formulation may include a balm, cream, or salve and an oxide constituent, and an antibiotic in equal parts. That is, the balm constituent, oxide constituent, and antibiotic may be in an equal parts 1:1:1 ratio as a premixed formulation.

The balm, cream, or salve may include at least one of petroleum, lanolin, paraffin wax, or 8-hydroxyquinoline sulfate 0.3%. More preferably, balm, cream, or salve may include petroleum, lanolin, paraffin wax, and 8-hydroxyquinoline sulfate 0.3%. In some embodiments, the balm, cream, or salve may be a preformulated mixture of petroleum, lanolin, paraffin wax, and 8-hydroxyquinoline sulfate 0.3%. The balm, cream, or salve may be constructed and arranged to moisten or soften the skin, prevent dry skin, commonly used for cuts, scrapes, allergies, and the like.

According to some embodiments, a multi-use healing formulation may include an oxide constituent such as, but not limited to, an oxide constituent including 40% zinc oxide such as a 40% zinc oxide rash cream.

According to some embodiments, a multi-use healing formulation may include an antibacterial constituent or topical antibiotic such as, but not limited to, bacitracin, mupirocin, gramicidin, or gentamycin. The antibiotic may be bacitracin zinc 500 units/gram topical ointment.

The multi-use healing formulation may be formulated for topical administration in the form of a cream applied to an injury site such as, but not limited to, burns, abrasions, lacerations, punctures, rashes, or avulsions. The multi-use healing formulation may be formulated to facilitate faster healing, particularly for burns and burn-related injuries.

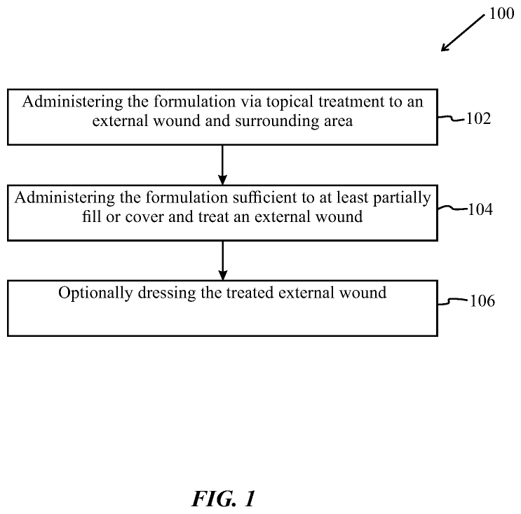

FIG. 1 depicts a simplified flowchart of a method 100 of administering the multi-use healing formulation according to step 102 in which the formulation may be administered to an external wound and the approximate surrounding area via a topical form of the multi-use healing formulation. According to step 104, the method 100 of administering the multi-use healing formulation may include applying the formulation sufficient to at least partially filled or cover and treat an external wound. According to step 104, the method 100 of administering the multi-use healing formulation may optionally include dressing the wound with an appropriate bandage, gauze, or the like.

The following description of variants is only illustrative of components, elements, acts, product, and methods considered to be within the scope of the invention and are not in any way intended to limit such scope by what is specifically disclosed or not expressly set forth. The components, elements, acts, product, and methods as described herein may be combined and rearranged other than as expressly described herein and still are considered to be within the scope of the invention.

Variation 1 may include multiple use cream formulation including a balm constituent; an oxide constituent; and an antibiotic.

Variation 2 may include a multiple use cream formulation as in variation 1, characterized in that multiple use cream formulation is formulated for topical administration.

Variation 3 may include a multiple use cream formulation as in any of variations 1 through 2, wherein the balm constituent, oxide constituent, and antibiotic are in an equal parts 1:1:1 ratio.

Variation 4 may include a multiple use cream formulation as in any of variations 1 through 3, wherein the oxide constituent includes 40% zinc oxide.

Variation 5 may include a multiple use cream formulation as in any of variations 1 through 4, wherein the oxide constituent including 40% zinc oxide is a 40% zinc oxide rash cream.

Variation 6 may include a multiple use cream formulation as in any of variations 1 through 5, wherein the antibiotic includes bacitracin.

Variation 7 may include a multiple use cream formulation as in any of variations 1 through 6, wherein the bacitracin includes bacitracin zinc 500 units/gram topical ointment.

Variation 8 may include a multiple use cream formulation as in any of variations 1 through 7, wherein the balm constituent includes at least one of petroleum, lanolin, paraffin wax, or 8-hydrocyquinoline sulfate 0.3%;

Variation 9 may include a multiple use cream formulation as in any of variations 1 through 8, wherein the balm constituent includes petroleum, lanolin, paraffin wax, and 8-hydrocyquinoline sulfate 0.3%.

Variation 10 may include a multiple use cream formulation as in any of variations 1 through 9, characterized in that multiple use cream formulation is formulated for treatment of an external wound.

Variation 11 may include a multiple use cream formulation as in any of variations 1 through 10, characterized in that multiple use cream formulation is formulated for topical administration to an at least one of a burn, abrasion, laceration, puncture, rash, or avulsion.

Variation 12 may include a multiple use cream formulation as in any of variations 1 through 11, characterized in that multiple use cream formulation is formulated to be administered as a topical formulation that at least partially fills a wound and wherein the wound is subsequently covered with a dressing.

According to variation 13, a method of administering a multiple use cream formulation may include at least partially disposing a formulation including a balm constituent; an oxide constituent; and an antibiotic on a surface characterized in that the multiple use cream formulation is formulated for treatment of at least one of a burn, abrasion, laceration, puncture, rash, or avulsion.

Variation 14 may include a method of administering a multiple use cream formulation as in variation 13, further including covering the external wound with at least one dressing.

Variation 15 may include a method of administering a multiple use cream formulation as in any of variations 13 through 14, wherein the balm constituent, oxide constituent, and antibiotic are in an equal parts 1:1:1 ratio.

Variation 16 may include a method of administering a multiple use cream formulation as in any of variations 13 through 15, wherein the oxide constituent includes 40% zinc oxide.

Variation 17 may include a method of administering a multiple use cream formulation as in any of variations 13 through 16, wherein the oxide constituent including 40% zinc oxide is a 40% zinc oxide rash cream.

Variation 18 may include a method of administering a multiple use cream formulation as in any of variations 13 through 17, wherein the antibiotic includes bacitracin zinc 500 units/gram topical ointment.

Variation 19 may include a method of administering a multiple use cream formulation as in any of variations 13 through 18, wherein the balm constituent includes at least one of petroleum, lanolin, paraffin wax, or 8-hydrocyquinoline sulfate 0.3%.

According to variation 20, a multiple use cream formulation may include a balm constituent including at least one of petroleum, lanolin, paraffin wax, or 8-hydrocyquinoline sulfate 0.3%; an oxide constituent including 40% zinc oxide; an antibiotic including bacitracin zinc 500 units/gram topical ointment; and wherein the balm constituent, oxide constituent, and antibiotic are in an equal parts 1:1:1 ratio and the multiple use cream formulation is characterized in that the multiple use cream formulation is formulated for treatment of an external wound.

According to variation 21, the composition includes 500 units of Bacitracin, 3.5 mg of Neomycin Sulfate, 10000 units of Polymyxin B Sulfate, 10 mg of Pramoxine HCl, and petroleum. Polysporin include 10000 units of Polymyxin B Sulfate, 0.25 mg of Gramicidin, and emulsifying wax, methylparaben, mineral oil, petroleum, poloxamer 188, propylene glycol, sodium hydroxide, sulfate acid and water.

In some embodiments, coconut oil is added to the composition.

In some embodiments, a sufficient amount of Silvadene is added to the composition to aid in burn recovery.

Many different embodiments have been disclosed herein, in connection with the above description and the drawings. It will be understood that it would be unduly repetitious and obfuscating to describe and illustrate every combination and subcombination of these embodiments. Accordingly, all embodiments can be combined in any way and/or combination, and the present specification, including the drawings, shall be construed to constitute a complete written description of all combinations and subcombinations of the embodiments described herein, and of the manner and process of making and using them, and shall support claims to any such combination or subcombination.

An equivalent substitution of two or more elements can be made for any one of the elements in the claims below or that a single element can be substituted for two or more elements in a claim. Although elements can be described above as acting in certain combinations and even initially claimed as such, it is to be expressly understood that one or more elements from a claimed combination can in some cases be excised from the combination and that the claimed combination can be directed to a subcombination or variation of a subcombination.

It will be appreciated by persons skilled in the art that the present embodiment is not limited to what has been particularly shown and described hereinabove. A variety of modifications and variations are possible in light of the above teachings without departing from the following claims.

I/we claim:

1. A multiple use cream formulation comprising:
a balm constituent;
an oxide constituent; and
an antibiotic.

2. A multiple use cream formulation as in claim 1, characterized in that multiple use cream formulation is formulated for topical administration.

3. A multiple use cream formulation as in claim 1 wherein the balm constituent, oxide constituent, and antibiotic are in an equal parts 1:1:1 ratio.

4. A multiple use cream formulation as in claim 1 wherein the oxide constituent comprises 40% zinc oxide.

5. A multiple use cream formulation as in claim 4 wherein the oxide constituent comprising 40% zinc oxide is a 40% zinc oxide rash cream.

6. A multiple use cream formulation as in claim 1 wherein the antibiotic comprises bacitracin.

7. A multiple use cream formulation as in claim 6 wherein the bacitracin comprises bacitracin zinc 500 units/gram topical ointment.

8. A multiple use cream formulation as in claim 1 wherein the balm constituent comprises at least one of petroleum, lanolin, paraffin wax, or 8-hydrocyquinoline sulfate 0.3%;

9. A multiple use cream formulation as in claim 1 wherein the balm constituent comprises petroleum, lanolin, paraffin wax, and 8-hydrocyquinoline sulfate 0.3%.

10. A multiple use cream formulation as in claim 1 according to claim 1, characterized in that multiple use cream formulation is formulated for treatment of an external wound.

11. A multiple use cream formulation as in claim 1, characterized in that multiple use cream formulation is formulated for topical administration to an at least one of a burn, abrasion, laceration, puncture, rash, or avulsion.

12. A multiple use cream formulation as in claim 1, characterized in that multiple use cream formulation is formulated to be administered as a topical formulation that at least partially fills a wound and wherein the wound is subsequently covered with a dressing.

13. A method of administering a multiple use cream formulation, comprising:
at least partially disposing a formulation comprising a balm constituent; an oxide constituent; and an antibiotic on a surface characterized in that the multiple use cream formulation is formulated for treatment of at least one of a burn, abrasion, laceration, puncture, rash, or avulsion.

14. A method of administering a multiple use cream formulation as in claim 13, further comprising covering the external wound with at least one dressing.

15. A method of administering a multiple use cream formulation as in claim 13, wherein the balm constituent, oxide constituent, and antibiotic are in an equal parts 1:1:1 ratio.

16. A method of administering a multiple use cream formulation as in claim 13, wherein the oxide constituent comprises 40% zinc oxide.

17. A method of administering a multiple use cream formulation as in claim 16, wherein the oxide constituent comprising 40% zinc oxide is a 40% zinc oxide rash cream.

18. A method of administering a multiple use cream formulation as in claim 13, wherein the antibiotic comprises bacitracin zinc 500 units/gram topical ointment.

19. A method of administering a multiple use cream formulation as in claim 13, wherein the balm constituent comprises at least one of petroleum, lanolin, paraffin wax, or 8-hydrocyquinoline sulfate 0.3%.

20. A multiple use cream formulation comprising:
a balm constituent comprising at least one of petroleum, lanolin, paraffin wax, or 8-hydrocyquinoline sulfate 0.3%;
an oxide constituent comprising 40% zinc oxide;
an antibiotic comprising bacitracin zinc 500 units/gram topical ointment; and
wherein the balm constituent, oxide constituent, and antibiotic are in an equal parts 1:1:1 ratio and the multiple use cream formulation is characterized in that the multiple use cream formulation is formulated for treatment of an external wound.
